(19) United States
(12) Patent Application Publication
(10) Pub. No.: US 2016/0151498 A1
(43) Pub. Date: Jun. 2, 2016

(54) Nanoparticle Compositions

(71) Applicant: Vuong Trieu, Agoura Hills, CA (US)

(72) Inventor: Vuong Trieu, Agoura Hills, CA (US)

(21) Appl. No.: 14/555,813

(22) Filed: Nov. 28, 2014

(51) Int. Cl. A61K 47/34; A61K 9/48; A61K 31/337

(52) U.S. Cl. CPC A61K 47/34; A61K 31/337; A61K 9/4816

(57) ABSTRACT

The present invention relates to compositions and methods of formulating nanoparticle drugs for cancer treatment in particular for intravenous administration in particular nanoparticle formulations containing cytotoxic drugs for the treatment of cancer. The compositions may have properties which facilitate the release of drugs into the patient including being unstable in plasma/blood, having low AUC, low Cₘₐₓ, high Vd, CMC above theoretical Cₘₐₓ of the drug, high tumor/plasma AUC. The present invention also provides for methods of administration and compositions which are unstable after administration to a patient so that the cytotoxic drug may bind to endogenous proteins such as albumin and be delivered to tumors in the patient.

### STATEMENT REGARDING FEDERALLY SPONSORED RESEARCH OR DEVELOPMENT

Not applicable.

### FIELD OF THE INVENTION

The present invention relates to compositions and methods of formulating nanoparticle drugs for cancer treatment in particular for intravenous administration in particular nanoparticle formulations containing cytotoxic drugs for the treatment of cancer. The compositions have properties which facilitate the release of drugs into the patient including being unstable in plasma/blood, having low AUC, low Cₘₐₓ, high Vd, CMC above theoretical Cₘₐₓ of the drug, high tumor/plasma AUC. The present invention also provides for methods of administration and compositions which are unstable after administration to a patient so that the cytotoxic drug may bind to endogenous proteins and be delivered to tumors in the patient.

### BACKGROUND OF THE INVENTION

Recent years have witnessed unprecedented growth of research and applications in the area of nanoscience and nanotechnology. There is increasing optimism that nanotechnology, as applied to medicine, will bring significant advances in the diagnosis and treatment of disease. Anticipated applications in medicine include drug delivery, both in vitro and in vivo diagnostics, nutraceuticals and production of improved biocompatible materials. Currently many substances are under investigation for drug delivery and more specifically for cancer therapy. Interestingly pharmaceutical sciences are using nanoparticles to reduce toxicity and side effects of drugs. Many drugs used to treat patients are administered by an intravenous route.

Abraxane® and Taxol® are chemotherapeutic drugs. Both drugs are used to treat solid tumours such as breast cancer and lung cancer. These cytotoxic medicines arrest the growth of cells in case of cancerous tissues. They essentially differ in the excipients they carry and their effectiveness. Paclitaxel is an antineoplastic drug used in chemotherapy. It is an alkaloid derived from plants and prevents microtubule formation in cells. The drug is solvent based and should be carefully administered since it is an irritant. The dosage and duration of administration of drug depends on the Body Mass Index. Side effects of Taxol include bone marrow suppression (primarily neutropenia), hair loss, arthralgias and myalgias, pain in the joints and muscles, peripheral neuropathy, nausea and vomiting, diarrhea, mouth sores, and hypersensitivity reaction, which can be dose limiting.

Abraxane® is paciltaxel formulated as albumin encapsulated nanoparticles. The Abraxane formulation is free of solvent—Cremophor—in Taxol®. The absence of solvent, allows the paclitaxel to bind to endogenous proteins and be transported by protein (ie. albumin) mediated transport mechanism. Protein receptors are common on the surface of tumor cells which facilitates the binding of drug molecule.

IG-001, also known as Genexol-PM or cCynviloq, is a Cremophor-free novel nanoparticle formulation of paclitaxel. IG-001 is a polymer bound nanoparticle paclitaxel. Instead of using biological polymer (ie. albumin as in Abraxane) to encapsulate the paclitaxel, IG-001 uses diblock mPEG-PDLLA polymer. Biodegradable polymeric micelle-type or nanoparticle drug compositions, containing a water-soluble amphiphilic block copolymer having a hydrophilic poly(alkylene oxide) component and a hydrophobic biodegradable component, have been used to develop formulations in which a hydrophobic drug is physically trapped in the micelle. This micelle-type composition, enveloping a hydrophobic drug, can solubilize the hydrophobic drug in a hydrophilic environment to form a solution.

Nanomedicine is an emerging field of medicine in which drug treatments can be improved by formulating new delivery systems for drugs without using solvents found in traditional formulations. However, many challenges must be overcome if the application of nanotechnology is to realize the anticipated improved understanding of the patho-physiological basis of disease, bring more sophisticated diagnostic opportunities, and yield improved therapies. New compositions and criteria for formulating nanoparticles which can be used for intravenous administration with a variety of drug components are essential to the progress of nanomedicine. In addition, while there are many cytotoxic drug compositions that are useful in the treatment of various cancers, there exists a need for formulating cancer drug compositions which can be easily administered and achieve maximum clinical effectiveness with low toxicity.

### SUMMARY OF THE INVENTION

The present invention relates to novel compositions and methods of treatment of patients using nanoparticle formulated drugs used for intravenous administration where the properties of the nanoparticle been engineered to achieved desired properties including certain pharmacokinetic (pK) parameters. The formulations of the present invention may provide drugs entrained by nanoparticles which have properties which are counterintuitive when compared to traditional formulations. For example an effective nanoparticle formulation for intravenous administration of drugs for the treatment of patients might be unstable in plasma/blood, provide for rapid drug release, exhibit low Cₘₐₓ, AUC and high Vd; characteristics of rapid tissue penetration.

The present invention also relates to methods of treatment of cancer patients with nanoparticle formulated cancer treatment drugs including cytotoxic drugs where the properties of the nanoparticle been engineered to achieved desired properties including certain pharmacokinetic parameters. The formulations of the present invention may provide cytotoxic drug entrained by nanoparticles which have properties which are counterintuitive when compared to traditional formulations. For example an effective nanoparticle formulation for intravenous administration of cytotoxic drugs might be unstable in plasma/blood, provide for rapid drug release, exhibit low Cₘₐₓ, AUC and high Vd; characteristics of rapid tissue penetration.

The present invention relates to cytotoxic drug compositions comprising a cytotoxic drug entrained in a nanoparticle where the composition has a critical micelle concentration (CMC) higher than the theoretical Cₘₐₓ.

The present invention relates to a cytotoxic drug composition comprising a cytotoxic drug entrained in a where the composition has a CMC higher than the Cₘₐₓ and where the composition may be bound to and transported by endogenous proteins such as albumin in a mammal.

The present invention relates to cytotoxic drug compositions comprising one or more cytotoxic drugs encapsulated in a diblock copolymer wherein the composition is has a critical micelle concentration (CMC) higher than the theoretical Cₘₐₓ.

The present invention relates to a cytotoxic drug composition comprising one or more cytotoxic drugs encapsulated in a diblock copolymer where the composition has a CMC higher than the theoretical Cₘₐₓ and where the composition may be bound to and transported by endogenous proteins such as albumin in a mammal.

The present invention also related to cytotoxic drug compositions comprising one or more cytotoxic drugs entrained in a nanoparticle where the composition has a low AUC or Cₘₐₓ or high Vd such that the composition is bound to and transported by endogenous proteins such as albumin when administered to a human.

The present invention also related to cytotoxic drug compositions comprising one or more cytotoxic drugs encapsulated in a diblock copolymer where the composition has a low AUC or Cₘₐₓ or high Vd such that the composition is bound to and transported by endogenous proteins such as albumin when administered to a human.

The present invention also relates to methods of administering the cytotoxic compositions of the present invention as well as administering these compositions in combination with other active cancer treating agents such as cisplatin/carboplatin and gemcitabine.

The present invention also relates to methods of administering the cytotoxic compositions of the present invention to treat breast cancer or pancreatic cancer or lung cancer or bladder cancer or ovarian cancer.

The present invention also relates to methods of determining the optimal formulation for a cytotoxic drug containing nanoparticle for the treatment of cancer including determining the optimal Cₘₐₓ, CMC, AUC and Vd.

### BRIEF DESCRIPTION OF THE FIGURES

FIG. 1a. Paclitaxel release from various formulations

FIG. 1b. Paclitaxel release evaluated by Rapid Equilibrium Dialysis

FIG. 2a. Dose-proportionality graph for IG-001, IG-002, Taxol™ and Abraxane

FIG. 2b. Volume of distribution graph for IG-001, IG-002, Taxol, and Abraxane

FIG. 3. Dissolution profile of IG-001

FIG. 4A. Size distribution of Genexol-PM nanparticles

FIG. 4B. Nanoparticle sizes versus concentration for Genexol-PM

### DETAILED DESCRIPTION OF THE INVENTION

The present invention relates to novel compositions for the treatment of patients using nanoparticle formulated drugs used for intravenous administration where the properties of the nanoparticle been engineered to achieved desired properties including certain pharmacokinetic (pK) parameters. The present invention relates to compositions and methods of formulating nanoparticle drugs for intravenous administration in particular nanoparticle formulations containing cytotoxic drugs for the treatment of cancer. The compositions may have properties which facilitate the release of drugs into the patient including being unstable in plasma/blood, having low AUC, low Cmax, high Vd, CMC above theoretical Cₘₐₓ of the drug, high tumor/plasma AUC. The present invention also provides for methods of administration and compositions which are unstable after administration to a patient so that the cytotoxic drug may bind to endogenous proteins such as albumin and be delivered to tumors in the patient.

Pharmacokinetics describes, quantitatively, the various steps of drug distribution in the body including the absorption of drugs, distribution of drugs to various organs and the elimination of drugs from the body. Various pharmacokinetic (pK) parameters include maximum observed plasma concentration (Cₘₐₓ), areas under the plasma concentration-time curve (AUCₗₐₛₜ and AUC_{inf}), areas under the first moment curve (AUMCₗₐₛₜ and AUMC_{inf}), time-to-maximum observed plasma concentration (Tₘₐₓ), half-life (T_{1/2}), the apparent terminal elimination rate constant (λ₂), and mean resident time (MRT).

Cₘₐₓ refers to the maximum concentration that a drug achieves in tested area after the drug has been administered. The Area Under the Curve (AUC) is a plot of concentration of drug in blood plasma against time. The area is computed from the time the drug is administered to the point where concentration in plasma is negligible. The Volume of Distribution (Vd) relates the amount of drug in the body to the measured concentration in the plasma. A large volume of distribution indicates that the drug distributes extensively into body tissues and fluids. Dose proportionality is also a common phrase used pharmacokinetics. Dose proportionality occurs when increases in the administered dose are accompanied by proportional increases in a measure of exposure like AUC or Cₘₐₓ. Thus an evaluation of dose proportionality usually includes exposure analysis of 3 or more doses to produce a graph. A discussion of various pharmacokinetic parameters and the methods of measuring them can be found in Clinical Pharmacokinetics and Pharmacodynamics: Concepts and Applications, M. Rowland and T. N. Tozer, (Lippincott, Williams & Wilkins, 2010).

Polymeric micelles and nanoparticles have been used in the delivery of various drugs. Micelle stability is influenced by various factors depending on the media environment including polymer concentration, molecular mass of the core-forming block, drug incorporation, other proteins and/or cells found in serum or blood. Stability of micelles depends on the polymer concentration. Polymer micelles have a critical micelle concentration (CMC) that is the lowest concentration of polymers to produce a micelle structure. Thus, micelles form when the concentration of the surfactant is greater than the critical micelle concentration and the temperature of the system is greater than the critical micelle temperature. Micelles can form spontaneously because of the balance between entropy an enthalpy. In aqueous systems, the hydrophobic effect is the driving force for micelle formulation and surfactant molecules assembling reduce the entropy. As the concentration of the lipid increases, the unfavorable entropy considerations from the hydrophobic end of the molecule prevail. At this point the lipid hydrocarbon chains of a portion of the lipids must be sequestered away from the water. Therefore, the lipid starts to form micelles. When surfactants are present above the CMC, they can act as emulsifiers that will allow a compound that is normally insoluble to dissolve. The CMC may be determined by a variety of methods including but not limited to: 1) spectroscopic measurements using a fluorescence probe, an absorbance dye 2 and other probes; 2) electrochemical measurement using electrophoresis or capillary electrophoresis; 3) surface tension measurements and contact angle measurement; 4) optical measurements using light scattering, optical fibers and refraction; 5) other methods such as ITC, chromatography, ultrasonic velocity and others. One method for determining CMC is by particle dissolution. Starting with a certain concentration (e.g. 5 mg/ml), the drug is serially diluted in a testing matrix (PBS, blood, plasma, etc.) and the size of the nanoparticle is determined by DLS. The concentration at which the nanoparticles disappear is the CMC.

It is commonly believed that effective intravenous nanoparticle formulations requires high blood/plasma level, stable nanoparticle (nanoparticle with critical micelles concentration below its Cₘₐₓ in blood/plasma) and slow release of drug, and would be effective in cancerous tumors. However, the compositions and methods of the present invention unexpected point to the opposite conclusion. Namely, effective nanoparticle formulations for intravenous administration of drugs, especially cytotoxic drugs are unstable in plasma/blood, provide for rapid drug release, exhibit low Cₘₐₓ, AUC and high Vd all of which are characteristics of rapid tissue penetration. The nanoparticle formulations of the present invention may be more effective or equally effective as conventional solvent based formulations at equal dosing.

The prior art teaches methods to prepare sustained release micelles in which polymers with very low CMC (<0.1 μg/ml) can be used for prolonging the circulation time before the micelle degrades. Upon intravenous injection, the micelles undergo dilution in the body. If the CMC of the micelles is high, the concentration of the polymer or surfactant falls below the CMC upon dilution and hence, the micelles dissociate. Therefore, the prior art teaches that a higher concentration of the polymer or surfactant has to be used to prepare the micelles or nanoparticles so that they withstand the dilution up to 5 L in the blood. However, the use of high concentrations might not be feasible due to toxicity related dose limitations. Therefore, the polymers are also selected for low CMC allowing for it to withstand dilution in blood. If the polymer or surfactant has a CMC lower than 0.1 μg/ml, concentrations such as 5 mg/ml may be used to prepare a micelle formulation in order to counter the dilution effects in the blood. A variety of polymers including diblock copolymers, triblock copolymers and graft copolymers have been synthesized for this purpose. Thus, the prior art teaches that the nanoparticles should be crafted to be stable even after intravenous administration. The compositions of the present invention provide for formulations in which the nanoparticles are less stable once administered such that the drug compound can be released from the nanoparticle. Release from the nanoparticle make the drug compound available to the endogenous proteins such as albumin delivery system. Nanoparticles of the present invention have CMC values which are higher than the Cₘₐₓ of the composition once delivered to a patient. In the nanoparticles of the present invention the CMC of the nanoparticles may be at least 10% higher than the expected Cₘₐₓ of the nanoparticle composition. In the nanoparticles of the present invention the CMC of the nanoparticles may be at least 20% higher or 25% higher or 30% higher or 35% higher or 40% higher or 45% higher or 50% higher or 55% higher or 60% higher or 65% higher or 70% higher or 75% higher or 80% higher or 85% higher or 90% higher or 95% higher or 100% higher or 125% higher or 150% higher or 175% higher or 200% higher or 500% than the expected Cₘₐₓ of the nanoparticle composition. In the nanoparticles of the present invention the CMC of the nanoparticles may be between about 10% higher to about 250% higher or about 10% higher to about 150% higher or about 10% higher to about 125% higher or about 10% higher to about 100% higher or from about 10% higher to about 90% higher or from about 10% higher to about 80% higher or from about 10% higher to about 70% higher or from about 10% higher to about 60% higher or from about 10% higher to about 50% higher or from about 10% higher to about 40% higher or from about 10% higher to about 30% higher or about 10% higher to about 20% higher or about 20% higher to about 125% higher or about 20% higher to about 100% higher or from about 20% higher to about 90% higher or from about 20% higher to about 80% higher or from about 20% higher to about 70% higher or from about 20% higher to about 60% higher or from about 20% higher to about 50% higher or from about 20% higher to about 40% higher or from about 50% higher to about 250% or from about 50% higher to about 125% higher or from about 50% higher to about 100% higher than the expected Cmax of the nanoparticle composition.

The nanoparticles of the present invention ideally release their contents in vivo but are stable in an iv bag, in an infusion solution or in a reconstitution vial. The nanoparticles of the present invention can be altered to accommodate the particular pK profile that is desirable for the drug to be delivered. The present invention relates to novel compositions and methods of treatment of patients using nanoparticle formulated drugs used for intravenous administration where the properties of the nanoparticle been engineered to achieved desired properties including one or more pharmacokinetic (pK) parameters.

The nanoparticles of the present invention may have low AUC and low Cₘₐₓ. In some embodiments the AUC of the nanoparticles of the present invention are at least 5% or at least 10% or at least 20% or at least 30% or at least 40% or at least 50% or at least 60% or at least 70% or at least 80% or at least 90% or at least 100% less than comparable solvent based formulations. In some embodiments the AUC of the nanoparticles of the present invention are between about 5% to about 100% or from about 5% to about 75% or from about 5% to about 50% or from about 5% to about 25% or from about 5% to about 10% or from about 10% to about 75% or from about 10% to about 50% or from about 10% to about 25% or from about 25% to about 100% or from about 25% to about 75% or from about 25% to about 50% less than comparable solvent based formulations.

In some embodiments the Cₘₐₓ (adjusted for dose and infusion rate) of the nanoparticles of the present invention are at least 5% or at least 10% or at least 20% or at least 30% or at least 40% or at least 50% or at least 60% or at least 70% or at least 80% or at least 90% or at least 100% less than comparable solvent based formulations. In some embodiments the AUC of the nanoparticles of the present invention are between about 5% to about 100% or from about 5% to about 75% or from about 5% to about 50% or from about 5% to about 25% or from about 5% to about 10% or from about 10% to about 75% or from about 10% to about 50% or from about 10% to about 25% or from about 25% to about 100% or from about 25% to about 75% or from about 25% to about 50% less than comparable solvent based formulations.

In some embodiments the Vd of the nanoparticles of the present invention the Vd of the nanoparticles may be at least 20% higher or 25% higher or 30% higher or 35% higher or 40% higher or 45% higher or 50% higher or 55% higher or 60% higher or 65% higher or 70% higher or 75% higher or 80% higher or 85% higher or 90% higher or 95% higher or 100% higher or 125% higher or 150% higher or 175% higher or 200% higher or 500% than the expected Vd of the solvent based composition.

In the nanoparticles of the present invention the CMC of the nanoparticles may be between about 10% higher to about 250% higher or about 10% higher to about 150% higher or about 10% higher to about 125% higher or about 10% higher to about 100% higher or from about 10% higher to about 90% higher or from about 10% higher to about 80% higher or from about 10% higher to about 70% higher or from about 10% higher to about 60% higher or from about 10% higher to about 50% higher or from about 10% higher to about 40% higher or from about 10% higher to about 30% higher or about 10% higher to about 20% higher or about 20% higher to about 125% higher or about 20% higher to about 100% higher or from about 20% higher to about 90% higher or from about 20% higher to about 80% higher or from about 20% higher to about 70% higher or from about 20% higher to about 60% higher or from about 20% higher to about 50% higher or from about 20% higher to about 40% higher or from about 50% higher to about 250% or from about 50% higher to about 125% higher or from about 50% higher to about 100% higher than the Cmax of the solvent based formulation.

The nanoparticles of the present invention also expected to increase the Overall Response Rate (ORR) of a given drug. ORR is defined as the proportion of patients whose best overall response is either complete response (CR) or partial response (PR) according to the standard called “Response Evaluation Criteria in Solid Tumors” (RECIST), and can be a measure of “effectiveness” of a drug. The compositions of the present invention are superior in ORR to currently existing formulations and compositions using the same cancer treatment drug.

Various formulations of nanoparticles are contemplated by the compositions of the present invention. Nanoparticles include but are not limited to dendrimers, polymer micelles, niosomes, nanogels, solid lipid nanoparticles, lipid nanostructured systems, cubosomes, liposomes, peptide nanotulules, metal colloids, carbon nanotubules, fullerenes, gold nanoparticles, gold nanoshells, silicon nanoparticles and magnetic colloids.

The nanoparticles of the present invention include colloidal dispersion systems which include macromolecule complexes, nanocapsules, microspheres, beads, and lipid-based systems including oil-in-water emulsions, micelles, mixed micelles, and liposomes. Liposomes, which are artificial membrane vesicles are useful as delivery vehicles in vivo. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidyl choline. Block polymers may be useful in formulating the nanoparticles of the present invention. One example is block copolymers with cyclodextrins which provide drug delivery as supramolecular polymeric micelles. This involves non-covalent interactions between a macromolecular polymer, which works as a host, and a small polymer molecule, which works as a guest. Triblock copolymer micelles are flower-like micelles can be formed with a triblock copolymer with small hydrophobic ends and a long hydrophilic midsection. When dissolved in water, such polymer molecules assemble to form flower-like micellar structure. These flower-like micelles can dissolve the drug in the hydrophobic core. Drug release was faster with crystalline PLA blocks than amorphous PLA blocks, possibly because crystalline PLA stacks together, leaving the drug largely at the periphery while amorphous PLA might better integrate/disperse the drug within the polymer matrix. Most micelle-forming polymers are first dissolved in organic solvent followed by addition to an aqueous medium to form micelles. The use of organic solvents can be avoided for some triblock copolymer micelles.

Furthermore, through suitable selection of polymers, greater drug loading as well as sustained drug release can be achieved. Freeze-dried micelles may be easily redispersable. Unimolecular micelles may also provide a mechanism for release of drugs. The unimolecular micelle is made out of a polymer that has several hydrophilic and hydrophobic portions in itself and forms a single molecular micelle. Lipids and PEG-like hydrophilic polymers can be conjugated to form such unimolecular micelles. One such polymer is core(laur) PEG. Multiarm block copolymers can also be used to formulate micelles. For instance star-shaped or multiarmed micelles can be formed with an amphiphilic block copolymer with multiple hydrophilic blocks and a single hydrophobic block. These polymers can form micelles if the number of arms is high enough. One such polymer is H40-PLA-mPEG. Graft polymers have recently attracted significant attention in preparing micelles. Cellulose graft polymers can be used to form micelles. The cellulose portion of the polymer can be the hydrophilic part, with any hydrophobic segment conjugated to it to form an amphiphilic graft polymer.

Polymers have some degree of toxicity even if they are biocompatible. Therefore, there is a need to synthesize materials that are more biocompatible for the preparation of micelles and incorporation of drugs. Oligopeptides can be very useful amphiphilic molecules for the preparation of micelles. Hydrophobic residues, such as alanine, can be used to synthesize the hydrophobic block and hydrophilic residues like histidine or lysine can be used to synthesize the hydrophilic block. Such molecules can be used as amphiphilic molecules to formulate micelles. A combination of polymer and polyamino acid can form an amphiphilic polymer. PEG-polyglutamic acid copolymer was used to prepare micelles.

The polymeric micelle nanoparticle formulations includes amphiphilic block copolymer which may comprise a hydrophilic block (A) and a hydrophobic block (B) linked with each other in the form of A-B, A-B-A or B-A-B structure. Additionally, the amphiphilic block copolymer may form core-shell type polymeric micelles in its aqueous solution state, wherein the hydrophobic block forms the core and the hydrophilic block forms the shell.

In one embodiment, the hydrophilic block (A) of the amphiphilic block copolymer may be polyethylene glycol (PEG) or monomethoxypolyethylene glycol (mPEG). Particularly, it may be mPEG. The hydrophilic block (A) may have a weight average molecular weight of 500-20,000 daltons, specifically 1,000-5,000 daltons, and more specifically 1,000-2,500 daltons.

The hydrophobic block (B) of the amphiphilic block copolymer may be a water-insoluble, biodegradable polymer. In one embodiment, the hydrophobic block (B) may be polylactic acid (PLA) or poly(lactic-co-glycolic acid) (PLGA). In another embodiment, the hydrophobic block (B) may have a weight average molecular weight of 500-20,000 daltons, specifically 1,000-5,000 daltons, and more specifically 1,000-2,500 daltons. Hydroxyl end groups of the hydrophobic block (B) may be protected with fatty acid groups, and particular examples of the fatty acid groups include acetate, propionate, butyrate, stearate, palmitate groups, and the like. The amphiphilic block copolymer comprising the hydrophilic block (A) and the hydrophobic block (B) may be present in the composition in an amount of 20-98 wt %, specifically 65-98 wt %, and more specifically 80-98 wt % based on the total dry weight of the composition.

In another embodiment, the hydrophilic block (A) and the hydrophobic block (B) may be present in the amphiphilic block copolymer in such a ratio that the copolymer comprises 40-70 wt %, specifically 50-60 wt % of the hydrophilic block (A) based on the weight of the copolymer. When the hydrophilic block (A) is present in a proportion less than 40%, the polymer has undesirably low solubility to water, resulting in difficulty in forming micelles. On the other hand, when the hydrophilic block (A) is present in a proportion greater than 70%, the polymer becomes too hydrophilic to form stable polymeric micelles, and thus the composition may not be used as a composition for solubilizing taxane.

A preferred paclitaxel formulation is IG-001 (also referred to as Genexol-PMT™, Cynviloq™) which is a Cremophor™-free, polymeric micelle formulation of paclitaxel. IG-001 comprises biodegradable di-block copolymer composed of methoxypoly (ethyleneglycol)-poly (lactide) to form nanoparticles with a paclitaxel-containing hydrophobic core, and a hydrophilic shell. Cynviloq is a polymeric formulation of paclitaxel in monomethoxy poly(ethylene glycol)-block-poly(D,L-lactide) (mPEG-PDLLA). Its Molecular Formula is: CH3O—[CH2 CH2O]m-CH2CH2O—[COCH(CH3)O]n-COCH(CH3)OH and has a molecular weight: Mn 3,000˜4,000. The molecular weight of the starting material; mPEG is 2000 g/mol and PDLLA is 144.3 g/mol.

mPEG-PDLLA is commercially manufactured in a 6 kg batch size by the reaction of methoxypolyethylene glycol (mPEG) with 3,6-dimethyl-1,4-dioxane-2,5-dione (D,L-lactide) in the presence of the catalyst stannous octoate (Sn(Oct)2) according to the equation shown in the figure below:

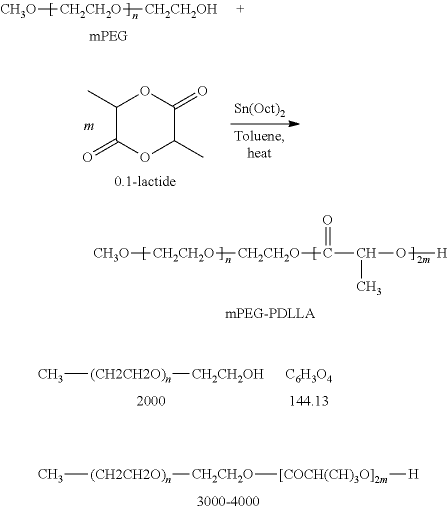

Micelles of mPEG-PDLLA containing paclitaxel may be made by melting the mPEG-PDLLA in a mixing tank over 30 min. After melting, paclitaxel and anhydrous ethanol are added and mixed for 30˜35 min. Ethanol is evaporated under vacuum for 120 min. Water for injection is added and mixed to produce micellar solution for 50±10 min. and then, the micellar solution is cooled. While the micellar solution is being prepared, anhydrous lactose and water for injection are added to another mixing tank, and mixed for 15±5 min. The lactose solution is cooled to under 5±3° C. The micellar solution is filtered through 0.45 Millipore filter into a mixing tank containing lactose solution. The filtered solution is mixed for 30±10 min. and the mixture is pressure-filtered with sterile nitrogen through 0.22 μm Millipak 100 filter. Before and after filtration and sterile filtration, integrity tests are performed to demonstrate that the filter assembly maintained its integrity throughout the entire filtration procedure. Filtered solution is filled into vials and filling volume is checked every 5 min. during the filling process. The filled vials are loaded into sterilized Freeze dryer and lyophilized. The capping process followed the lyophilization process and the capped vials are inspected for foreign matter.

The size of the nanoparticles for the IG-001 is a Gaussian distribution where the mean particle size is about 10 nm to about 50 nm or from about 10 nm to about 40 nm or from about 10 nm to about 30 nm or from about 10 nm to about 25 nm or from about 10 nm to about 20 nm, or from about 15 nm to 50 nm, or from about 15 nm to about 40 nm or from about 15 nm to about 30 nm or from about 15 nm to about 25 nm or from about 15 nm to about 20 nm or from about 20 nm to about 50 nm or from about 20 nm to about 40 nm, or from about 20 nm to about 30 nm or from about 20 nm to about 25 nm. In a preferred embodiment the micelles are from about 22 nm to about 26 nm. Micelles are monodisperse with a polydispersity index of approximately 0.13 (range from about 0.120 to 0.145). Their sizes maintained constant regardless of paclitaxel concentration in the micelle solution. There appears to be no appreciable time and temperature-dependent change in particle size of the micelles at various concentrations when diluted with 0.9% NaCl solution. The loading percentage of the paclitaxel is about and The loading efficiency of paclitaxel is from about 95.0% to about 99.9%, or from about 95.0% to about 99.5% or from about 95.0% to about 99.0%, or from about 95.0% to about 98.5%, or from about 95.0% to about 98.0%, or from about 95.0% to about 97.5%, or from about 95.0% to about 97.0%, or from about 95.0% to about 96.5% or from 96.0% to about 99.9%, or from about 96.0% to about 99.5% or from about 96.0% to about 99.0%, or from about 96.0% to about 98.5%, or from about 96.0% to about 98.0%, or from about 96.0% to about 97.5%, or from about 96.0% to about 97.0%, or from about 96.0% to about 96.5% or from 97.0% to about 99.9%, or from about 97.0% to about 99.5% or from about 97.0% to about 99.0%, or from about 97.0% to about 98.5%, or from about 97.0% to about 98.0%, or from about 97.0% to about 97.5%.

The critical micelle concentration is of this embodiment of the invention is about 0.007 mg/mL. as measured by adding the copolymer to aqueous solutions of pyrene and determining the concentration that caused a change in the pyrene fluorescence wavelength.

The polymeric micelles in aqueous solution of Genexol-PM showed surface charges ranging from −2.0 mV to +1.0 mV which are considered almost neutral. Their surface charges were almost constant regardless of paclitaxel concentration in Genexol-PM solution.

Cytotoxic drugs entrained or encapsulated in the nanoparticles of the present invention may include but are not limited to carboplatin, cisplatin, cyclophoshaminde, doxorubicin, etoposide, fluoruracil, gemcitabine, irinotecan, methotrexate, topotecan, vincristine, vinblastine, docetaxel, paclitaxel, 7-epipaclitaxel, t-acetyl paclitaxel, 10-desacetyl-paclitaxel, 10-desacetyl-7-epipaclitaxel, 7-xylosylpaclitaxel, 10-desacetyl-7-glutarylpaclitaxel, 7-N,N-dimethylglycylpaclitaxel, 7-L-alanylpaclitaxel, epothilone, 17-AAG, or rapamycin.

Cancer types for which the methods of the present invention may be useful include but are not limited to bladder cancer, ovarian cancer, breast cancer, pancreatic cancer, liver cancer, non-small cell lung cancer (NSCLC) and other lung cancers.

Other intravenous drugs suitable for administration with the nanoparticles of the present invention include but are not limited to those for the treatment of infectious disease, cancer and proliferative disease.

Intravenous infusions of taxanes such as paclitaxel into cancer patients result in hypersensitivity reactions in such patients. The present invention relates to methods of treating cancer patients with paclitaxel whereby the hypersensitivity reactions induced by such treatments are reduced. The present invention relates to methods of treating cancer patients with paclitaxel infusions in which the infusion time period is reduced thereby reducing the number of hypersensitivity reactions. The infusion times for treatment of cancer patients with taxanes can be less than 4 hours, less than 3 hours, or less than 2.5 hours, or less than 2 hours or less than 1.5 hours or less than 1 hour or less than 45 minutes or less than 30 minutes. The infusion times for treatment of cancer patients with taxanes can be about 15 minutes to about 3 hours, or from about 15 minutes to about 2.5 hours, or from about 15 minutes to about 2 hours, or from about 15 minutes to about 1.5 hours, or from about 15 minutes to about 1 hour, or from about 15 minutes to about 45 minutes, or from about 15 minutes to about 30 minutes, or from about 30 minutes to about 3 hours, or from about 30 minutes to about 2.5 hours, or from about 30 minutes to about 2 hours, or from about 30 minutes to about 1.5 hours, or from about 30 minutes to about 1 hour, or from about 30 minutes to about 45 minutes, or from about 45 minutes to about 3 hours, or from about 45 minutes to about 2.5 hours, or from about 45 minutes to about 2 hours, or from about 45 minutes to about 1.5 hours, or from about 45 minutes to about 1 hour, or from 1 hour to about 3 hours, or from about 1 hour to about 2.5 hours, or from about 1 hour to about 2 hours, or from about 1 hour to about 1.5 hours, or from about 1.5 hours to about 3 hours, or from about 1.5 hours to about 2.5 hours, or from about 1.5 hours to about 2 hours.

The present invention relates to methods of treating cancer patients with taxanes whereby the hypersensitivity reactions induced by such treatments are reduced. The present invention relates to methods of treating cancer patients with taxane infusions in which the concentration of the taxane infused into the patient is controlled to reduce the number of hypersensitivity reactions. The concentration of the paclitaxel infused into the patient can be greater than about 0.6 mg/ml, or about 0.7 mg/ml, or about 0.8 mg/ml, or about 0.9 mg/ml, or about 1.0 mg/ml, or about 1.1 mg/ml, or about 1.2 mg/ml, or about 1.3 mg/ml, or about 1.4 mg/ml, or about 1.5 mg/ml, or about 1.6 mg/ml, or about 1.7 mg/ml, or about 1.8 mg/ml, or about 1.9 mg/ml, or about 2.0 mg/ml, or about 2.1 mg/ml, or about 2.2 mg/ml, or about 2.3 mg/ml, or about 2.4 mg/ml, or about 2.5 mg/ml, or about 2.6 mg/ml, or about 2.7 mg/ml, or about 2.8 mg/ml, or about 2.9 mg/ml or about 3.0 mg/ml, or about 3.1 mg/ml, or about 3.2 mg/ml, or about 3.3 mg/ml, or about 3.4 mg/ml, or about 3.5 mg/ml, or about 3.6 mg/ml, or about 3.7 mg/ml, or about 3.8 mg/ml, or about 3.9 mg/ml or about 4.0 mg/ml, or about 4.1 mg/ml, or about 4.2 mg/ml, or about 4.3 mg/ml, or about 4.4 mg/ml, or about 4.5 mg/ml, or about 4.6 mg/ml, or about 4.7 mg/ml, or about 4.8 mg/ml, or about 4.9 mg/ml or about 5.0 mg/ml, or about 5.1 mg/ml, or about 5.2 mg/ml, or about 5.3 mg/ml, or about 5.4 mg/ml, or about 5.5 mg/ml, or about 5.6 mg/ml, or about 5.7 mg/ml, or about 5.8 mg/ml, or about 5.9 mg/ml or about 6.0 mg/ml, or about 6.1 mg/ml, or about 6.2 mg/ml, or about 6.3 mg/ml, or about 6.4 mg/ml, or about 6.5 mg/ml, or about 6.6 mg/ml, or about 6.7 mg/ml, or about 6.8 mg/ml, or about 6.9 mg/ml, or about 7.0 mg/ml, or about 7.1 mg/ml, or about 7.2 mg/ml, or about 7.3 mg/ml, or about 7.4 mg/ml, or about 7.5 mg/ml, or about 7.6 mg/ml, or about 7.7 mg/ml, or about 7.8 mg/ml, or about 7.9 mg/ml or about 8.0 mg/ml, or about 8.1 mg/ml, or about 8.2 mg/ml, or about 8.3 mg/ml, or about 8.4 mg/ml, or about 8.5 mg/ml, or about 8.6 mg/ml, or about 8.7 mg/ml, or about 8.8 mg/ml, or about 8.9 mg/ml or about 9.0 mg/ml, or about 9.1 mg/ml, or about 9.2 mg/ml, or about 9.3 mg/ml, or about 9.4 mg/ml, or about 9.5 mg/ml, or about 9.6 mg/ml, or about 9.7 mg/ml, or about 9.8 mg/ml, or about 9.9 mg/ml, or about 10.0 mg/ml. The concentration of the taxane infused into the patient can be about 0.6 mg/ml to about 10.0 mg/ml, or from about 0.6 mg/ml to about 8.0 mg/ml, or from about 0.6 mg/ml to about 7.0 mg/ml, or from about 0.6 mg/ml to about 6.0 mg/ml, or from about 0.6 mg/ml to about 5.0 mg/ml, or from about 0.6 mg/ml to about 4.0 mg/ml, or from about 0.6 mg/ml to about 3.0 mg/ml, or from about 0.6 mg/ml to about 2.0 mg/ml, or from about 0.7 mg/ml to about 1.0 mg/ml, of from about 0.7 mg/ml to about 10.0 mg/ml, or from about 0.7 mg/ml to about 8.0 mg/ml, or from about 0.7 mg/ml to about 7.0 mg/ml, or from about 0.7 mg/ml to about 6.0 mg/ml, or from about 0.7 mg/ml to about 5.0 mg/ml, or from about 0.7 mg/ml to about 4.0 mg/ml, or from about 0.7 mg/ml to about 3.0 mg/ml, or from about 0.7 mg/ml to about 2.0 mg/ml, or from about 0.7 mg/ml to about 1.0 mg/ml, or from about 0.8 mg/ml to about 10.0 mg/ml, or from about 0.8 mg/ml to about 8.0 mg/ml, or from about 0.8 mg/ml to about 7.0 mg/ml, or from about 0.8 mg/ml to about 6.0 mg/ml, or from about 0.8 mg/ml to about 5.0 mg/ml, or from about 0.8 mg/ml to about 4.0 mg/ml, or from about 0.8 mg/ml to about 3.0 mg/ml, or from about 0.8 mg/ml to about 2.0 mg/ml, or from about 0.8 mg/ml to about 1.0 mg/ml, of from about 0.9 mg/ml to about 10.0 mg/ml, or from about 0.9 mg/ml to about 8.0 mg/ml, or from about 0.9 mg/ml to about 7.0 mg/ml, or from about 0.9 mg/ml to about 6.0 mg/ml, or from about 0.9 mg/ml to about 5.0 mg/ml, or from about 0.9 mg/ml to about 4.0 mg/ml, or from about 0.9 mg/ml to about 3.0 mg/ml, or from about 0.9 mg/ml to about 2.0 mg/ml, or from about 0.9 mg/ml to about 1.0 mg/ml, of from about 1.0 mg/ml to about 10.0 mg/ml, or from about 1.0 mg/ml to about 8.0 mg/ml, or from about 1.0 mg/ml to about 7.0 mg/ml, or from about 1.0 mg/ml to about 6.0 mg/ml, or from about 1.0 mg/ml to about 5.0 mg/ml, or from about 1.0 mg/ml to about 4.0 mg/ml, or from about 1.0 mg/ml to about 3.0 mg/ml, or from about 1.0 mg/ml to about 2.0 mg/ml, or from about 1.25 mg/ml to about 10.0 mg/ml, or from about 1.25 mg/ml to about 8.0 mg/ml, or from about 1.25 mg/ml to about 7.0 mg/ml, or from about 1.25 mg/ml to about 6.0 mg/ml, or from about 1.25 mg/ml to about 5.0 mg/ml, or from about 1.25 mg/ml to about 4.0 mg/ml, or from about 1.25 mg/ml to about 3.0 mg/ml, or from about 1.25 mg/ml to about 2.0 mg/ml, or from about 1.5 mg/ml to about 10.0 mg/ml, or from about 1.5 mg/ml to about 8.0 mg/ml, or from about 1.5 mg/ml to about 7.0 mg/ml, or from about 1.5 mg/ml to about 6.0 mg/ml, or from about 1.5 mg/ml to about 5.0 mg/ml, or from about 1.5 mg/ml to about 4.0 mg/ml, or from about 1.5 mg/ml to about 3.0 mg/ml, or from about 1.5 mg/ml to about 2.0 mg/ml, or about 2.0 mg/ml to about 10.0 mg/ml, or from about 2.0 mg/ml to about 8.0 mg/ml, or from about 2.0 mg/ml to about 7.0 mg/ml, or from about 2.0 mg/ml to about 6.0 mg/ml, or from about 2.0 mg/ml to about 5.0 mg/ml, or from about 2.0 mg/ml to about 4.0 mg/ml, or from about 2.0 mg/ml to about 3.0 mg/ml, or from about 2.5 mg/ml to about 10.0 mg/ml, or from about 2.5 mg/ml to about 8.0 mg/ml, or from about 2.5 mg/ml to about 7.0 mg/ml, or from about 2.5 mg/ml to about 6.0 mg/ml, or from about 2.5 mg/ml to about 5.0 mg/ml, or from about 2.5 mg/ml to about 4.0 mg/ml, or from about 2.5 mg/ml to about 3.0 mg/ml, or from about 3.0 mg/ml to about 10.0 mg/ml, or from about 3.0 mg/ml to about 8.0 mg/ml, or from about 3.0 mg/ml to about 7.0 mg/ml, or from about 3.0 mg/ml to about 6.0 mg/ml, or from about 3.0 mg/ml to about 5.0 mg/ml, or from about 3.0 mg/ml to about 4.0 mg/ml, or from about 3.5 mg/ml to about 10.0 mg/ml, or from about 3.5 mg/ml to about 8.0 mg/ml, or from about 3.5 mg/ml to about 7.0 mg/ml, or from about 3.5 mg/ml to about 6.0 mg/ml, or from about 3.5 mg/ml to about 5.0 mg/ml, or from about 3.5 mg/ml to about 4.0 mg/ml, or from about 4.0 mg/ml to about 10.0 mg/ml, or from about 4.0 mg/ml to about 8.0 mg/ml, or from about 4.0 mg/ml to about 7.0 mg/ml, or from about 4.0 mg/ml to about 6.0 mg/ml, or from about 4.0 mg/ml to about 5.0 mg/ml, or from about 4.5 mg/ml to about 10.0 mg/ml, or from about 4.5 mg/ml to about 8.0 mg/ml, or from about 4.5 mg/ml to about 7.0 mg/ml, or from about 4.5 mg/ml to about 6.0 mg/ml, or from about 4.5 mg/ml to about 5.0 mg/ml, or from about 5.0 mg/ml to about 10.0 mg/ml, or from about 5.0 mg/ml to about 8.0 mg/ml, or from about 5.0 mg/ml to about 7.0 mg/ml, or from about 5.0 mg/ml to about 6.0 mg/ml, or from about 5.5 mg/ml to about 10.0 mg/ml, or from about 5.5 mg/ml to about 8.0 mg/ml, or from about 5.5 mg/ml to about 7.0 mg/ml, or from about 5.5 mg/ml to about 6.0 mg/ml, or from about 6.0 mg/ml to about 10.0 mg/ml, or from about 6.0 mg/ml to about 8.0 mg/ml, or from about 6.0 mg/ml to about 7.0 mg/ml.

The PK parameters of paclitaxel from Genexol-PM studies demonstrated a low degree of variability and increased dose proportionally over the dose range tested and were quite different from paclitaxel. The mean paclitaxel concentration-time profiles following an infusion of Genexol-PM were characterized by a pronounced distribution phase followed by the terminal elimination phase. Within 5 to 15 minutes of the end of the infusion, paclitaxel concentrations dropped to one-half or one-third of the peak level. Constant-rate infusions of nanoparticle compositions of the present invention provide a maximum plasma paclitaxel concentrations between about 1.0 hour to about 4.0 hours, or from about 1.0 hours to about 3.5 hours, or from about 1.0 hour to about 3.0 hours, or from about 1.0 hours to about 2.5 hours or from about 1.0 hour to about 2.0 hours, or from about 1.5 hours to about 4.0 hours or from about 1.5 hour to about 3.5 hours, or from about 1.5 hours to about 3.0 hours or from about 1.5 hour to about 2.5 hours, or from about 1.5 hours to about 2.0 hours. Values of Cmax ranged from about 500 to about 1000, or from about 500 to about 900, or from about 500 to about 800, or from about 500 to about 750, or from about 500 to about 700, or from about 500 to about 650, or from about 600 to about 1000, or from about 600 to about 900, or from about 600 to about 800, or from about 600 to about 700 after 85 mg/m2 infusion. Values of Cmax ranged from about 4000 to about 8000, or from about 4000 to about 7000, or from about 4000 to about 6000, or from about 4000 to about 5000, or from about 5000 to about 8000, or from about 5000 to about 7000, or from about 5000 to about 6000 ng/ml for a 390 mg/m2 infusion. The AUC values for an infusion of 300 mg/m² paclitaxel for nanoparticle compositions of the present invention were about 5000 to about 15000, or from about 5000 to about 14000, or from about 5000 to about 13000 or from about 5000 to about 12000 or from about 5000 to about 11000, or from about 5000 to about 10000, or from about 5000 to about 9000 or from about 5000 to about 8000 or from about 6000 to about 15000, or from about 6000 to about 14000, or from about 6000 to about 13000 or from about 6000 to about 12000 or from about 6000 to about 11000, or from about 6000 to about 10000, or from about 6000 to about 9000 or from about 6000 to about 8000 or from about 7000 to about 15000, or from about 7000 to about 14000, or from about 7000 to about 13000 or from about 7000 to about 12000 or from about 7000 to about 11000, or from about 7000 to about 10000, or from about 7000 to about 9000 ng·hr/ml. The mean values of total systemic clearance of paclitaxel following 3-hour infusions of Genexol-PM were 12.1-33.3 L/hr/m2. These values found to be somewhat higher than the systemic total body clearance published in the literature for Taxol (12.2 to 17.7 L/hr/m2).4 The mean Vd of paclitaxel in the terminal elimination phase following infusions of Genexol-PM ranged from 328 to 897 L/m2, which is significantly greater than the Vd of paclitaxel reported for Taxol (67 to 182 L/m²: estimated from mean values of CL and T_{1/2}).

### EXAMPLES

### Example 1

### Paclitaxel Release

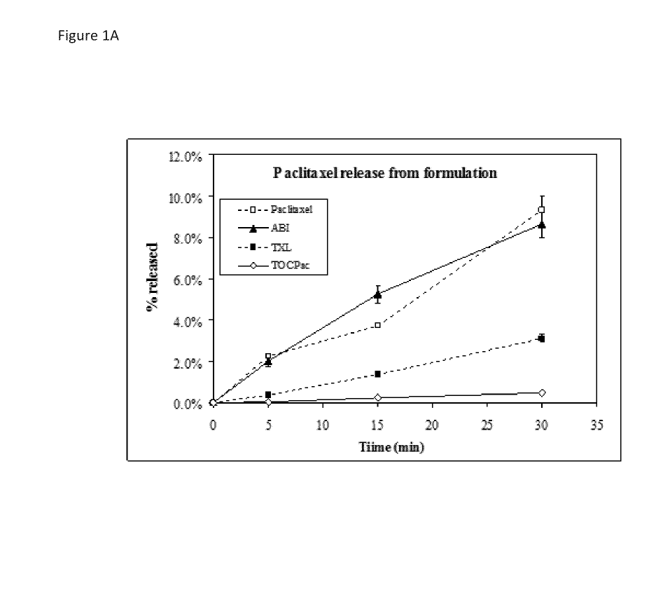

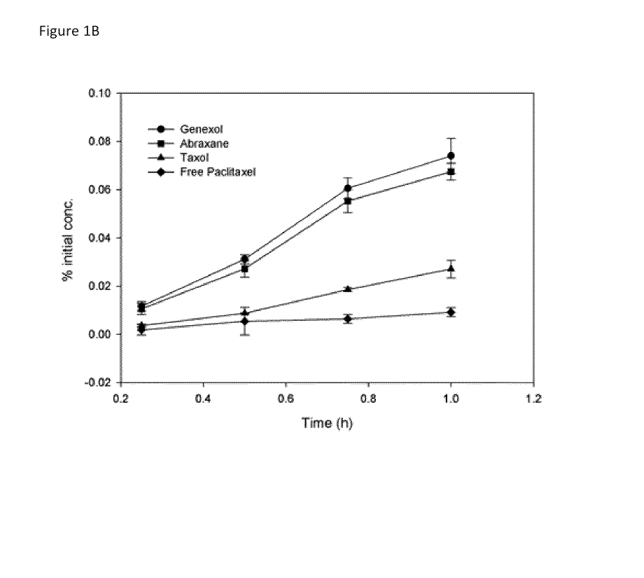

Paclitaxel release from each formulation was tested using equilibrium dialysis. Briefly, paclitaxel, IG-001, IG-002 (Tocosol-Pac), Taxol or reconstituted Abraxane (ABI) was added to one side of the well, and blank buffer to the other side. Samples were taken from the buffer side for the analysis of the appearance of free paclitaxel. The drug release profile from Abraxane appears similar to neat paclitaxel. Drug release is slowest for IG-002 (0.5% at 30 minutes, statistically significant versus the other three groups), followed by Taxol. Fast release was found for IG-001 and Abraxane. Results are shown in FIG. 1a and 1b.

### Example 2

### Pharmacokinetics of Unstable Nanoparticles

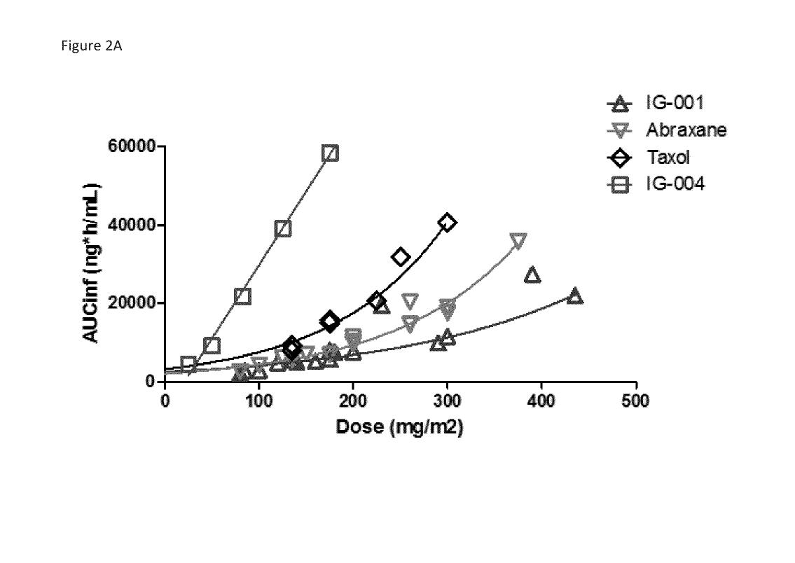

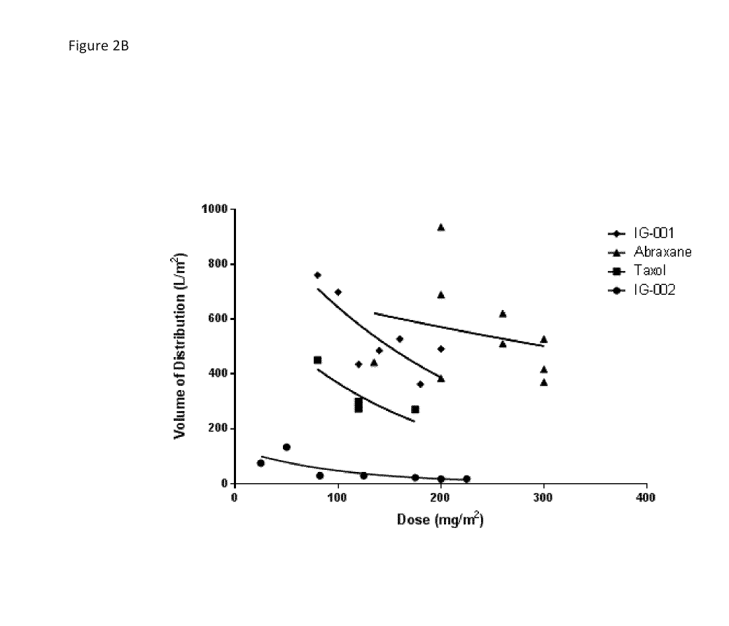

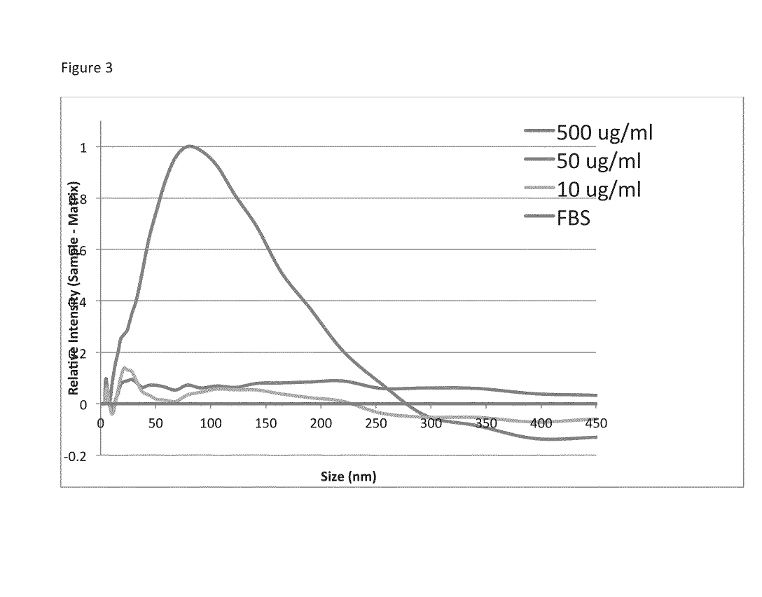

Clinical pharmacokinetics of IG-001 (Genexol-PM) was compared to Taxol and Abraxane and IG-002 (Tocosol). IG-001 range for PK dose-proportionality is the most expanded of the four paclitaxel formulations examined (Taxol, Abraxane, IG-001, IG-002) (FIG. 2a). Abraxane PK deviated from proportionality above 300 mg/m²; whereas IG-001 PK remained dose-proportional up to the highest dose of 435 mg/m². Additionally, the unstable nanoparticles IG-001 and Abraxane have lower AUC across all dose levels in comparison to the stable nanoparticle—IG-002/Tocosol.

Volume of distribution—Vd was higher for the unstable nanoparticles Abraxane and IG-001 versus stable nanoparticle (Tocosol/IG-002) or the solvent based paclitaxel formulation (Taxol). FIG. 2b.

### Example 3

### Dissolution in Serum

IG-001 (Genexol-PM) is a Cremophor-free, polymeric micelle formulation of paclitaxel utilizing biodegradable di-block copolymer composed of methoxy poly (ethylene glycol)-poly (lactide) to form nanoparticles with paclitaxel containing a hydrophobic core and a hydrophilic shell. IG-001 has a mean diameter of 25 nm with relatively low light scattering potential. IG-001 rapidly dissociates from intact nanoparticles upon dilution in serum at concentrations less than 50 ug/ml—higher than the Cₘₐₓ of IG-001—following a 3 hr infusion (FIG. 10). The CMC is higher than theoretical maximum drug level. Therefore, once administered, IG-001 readily gives up its paclitaxel cargo to endogenous proteins such as albumin for transport into the underlying tissues.

### Example 4

### Particle Size and Size Distribution

Reconstituted solutions of Cynviloq were prepared by dilution with 0.9% saline to make 0.6, 3.0, 5.0, or 6.0 mg/mL of paclitaxel. Particle size and size distribution of micelles was determined by dynamic light scattering spectrometer at room temperature.

Changes in particle size were investigated at 0, 4, 8, and 24 h after storage at room temperature (20′25° C.) and at 0, 24, 48, 72 h after storage at 2 to 8° C. Each measurement was repeated three times.

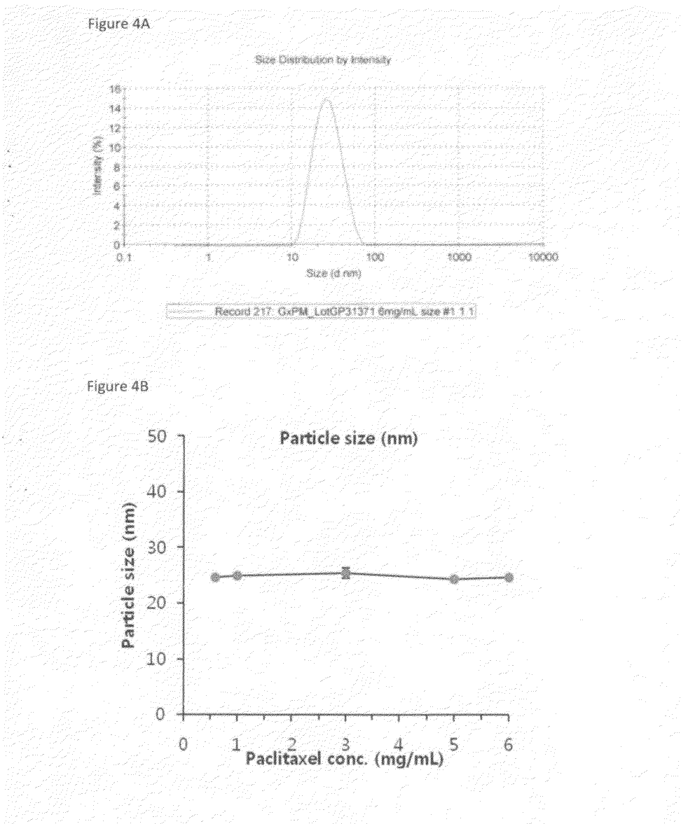

The micelles in aqueous solution had particle sizes ranging from 22 to 26 nm when measured by dynamic light scattering analysis. 4A shows that the micelles are monodisperse with a polydispersity index of approximately 0.13. Their sizes maintained constant regardless of paclitaxel concentration in the micelle solution as shown in FIG. 4B. There was no appreciable time and temperature-dependent change in particle size of the micelles at various concentrations when diluted with 0.9% NaCl solution, as shown in Table 1. Regarding the between-lot variation of particle sizes, no significant difference was observed among three lots

TABLE 1
Time and temperature-dependent changes in particle size of micelles
at various concentrations when diluted with 0.9% NaCl solution.

| | Temp (° C.) | | | | | | | |
|---|---|---|---|---|---|---|---|---|
| | 25 ± 2 | | | | 5 ± 3 | | | |
| | Time (h) | | | | | | | |
| | 0 | 4 | 8 | 24 | 0 | 24 | 48 | 72 |
| Concentration | Particle Size (nm) | | | | | | | |
| 0.6 mg/mL | 24.06 | 22.62 | 22.15 | 22.24 | 24.06 | 23.48 | 22.82 | 23.01 |
| 3.0 mg/mL | 23.25 | 21.50 | 21.18 | 21.23 | 23.25 | 22.29 | 22.05 | 22.02 |
| 5.0 mg/mL | 22.33 | 20.92 | 20.45 | 20.44 | 22.33 | 21.57 | 21.29 | 21.36 |

Particle size and polydispersity of micelles in
three IG-001 lots at 6 mg paclitaxel/mL

| Lot | Particle size (nm) Mean ± SD | Polydispersity, Mean ± SD |
|---|---|---|
| GP31371 | 25.43 ± 0.11 | 0.145 ± 0.004 |
| GP313B1 | 22.26 ± 0.22 | 0.120 ± 0.020 |
| GP31431 | 23.59 ± 0.20 | 0.130 ± 0.020 |

### Example 5

### Drug Loading and Encapsulation Efficiency

After estimating the paclitaxel content using HPLC analysis, loading percentage and loading efficiency were calculated using the following equations:

Loading percentage (%)=a/(b+c)×100%

Loading efficiency (%)=a/b×100%

Where a is the amount of paclitaxel loaded in micelles, b is the amount of paclitaxel used in preparation of the micelles, c is the amount of copolymer used in preparation of the micelles.

Loading percentage and loading efficiency of paclitaxel determined from this study are provided in the table below. If 100% of paclitaxel were loaded into the micelles, the loading percentage would be approximately 16.67%. The data show that the maximum amount of free paclitaxel is approximately 0.2%.

TABLE 2
Loading percentages and loading efficiencies
of paclitaxel in three lots

| Drug Product | Loading percentage (%) | Loading efficiency (%) |
|---|---|---|
| Batch | Mean ± SD | Mean ± SD |
| GP31371 | 16.48 ± 0.30 | 99.40 ± 1.61 |
| GP313B1 | 16.32 ± 0.33 | 97.93 ± 2.01 |
| GP31431 | 16.55 ± 0.29 | 99.28 ± 1.72 |

### Example 6

### Stability

Forced degradation studies were performed on Genexol-PM under the condition of dry heat, hydrolysis, oxidation, and photolysis, as defined by ICH*. For thermal stress testing, the drug powder was sealed in glass ampoules and heated in dry-bath at at 60° C. for 21 days. Base degradation was performed by incubating 30 mL of Genexol-PM solution (1 mg paclitaxel/mL) with 4.5 ml of 0.05 N NaOH for 4 hous. Acid degradation was performed by incubating 30 mL of Genexol-PM solution (1 mg paclitaxel/mL) with 6.0 mL of 5.0N HCl for 24 hours. Oxidative degradation was performed by incubation of 30 mL of Genexol-PM solution (1 mg paclitaxel/mL) diluted with 3.0 ml of 30% hydrogen peroxide for 30 hours.

The photolytic studies were carried out in solid state by spreading a thin layer of drug in a glass vial and exposing it directly to the combination of UV (NLT 1.2 watt hours/m²) and florescent light (NLT 1.2 million Lux hours) for 8 days or florescent light only in a photo stability chamber set for 15 days. The samples were exposed by UV and fluorescent lamp simultaneously for the initial 8 days. Thereafter, they were exposed only by fluorescent lamp. A parallel set was kept in dark under similar conditions as a control.

The HPLC instrument employed was Agilent 1100 series (manufactured by Agilent Technologies, Waldbronn, Germany) LC system with variable wavelength detector (VWD and also a diode array detector (DAD). The out put signal was monitored and processed using Chemstation Software (designed by Agilent Technologies, Waldbronn, Germany).

The paclitaxel content in Genexol-PM was measured using HPLC. Briefly, samples were analyzed by isocratic HPLC on a system consisting of a binary pump, a Zorbax eclipse column (5 um, 4.6×150 mm, Agilent, USA) using a flow of 1.5 mL/min and UV/Visible detection (λ=227 nm) with a lower detection limit of 10 ng/mL. The mobile phase was a mixture of 45% acetonitrile and 55% deionized water (v/v).

The impurities in Genexol-PM were analyzed by gradient HPLC using 150×4.6 mm Agilent Eclipse XDB C18 5 μm analytical column. The mobile phase consisted of water and acetonitrile and was pumped at an isocratic flow of 1 ml/min at room temperature with the following gradient condition:

(0˜30 min) water:acetonitrile=65:35→35:65

(30˜33 min) water:acetonitrile=35:65

(33˜35 min) water:acetonitrile=35:65→65:35

(35˜40 min) water:acetonitrile=65:35

The PDA detection wave length was set at 227 nm. All separations were performed at ambient temperature.

All the degradation products formed during forced decomposition studies were well separated from the analyte peak demonstrating that the developed method was specific and stability-indicating. Peak puriy test results confirmed that the paclitaxel peak is homogenous and pure in all stress samples, analyzed under DAD (Table 4-10).

### Base Degradation

Dissolution of Genexol-PM in basic aqueous solutions results in the rapid formation of several degradants. Paclitaxel content was found to decrease from 99.8% to 87.0% rapidly only for about 4 hours even at a very low concentration of NaOH solution (0.0075 N). More than eleven impurities were increased by base-induced degradation. Among them, the impurity at RRT 1.28 had the most prominent peak area which increased from 0% to 9.2% only for 4 hours (FIG. 4-11). The impurity at RRT 1.05 was also significantly increased from 0% to 2.1% for 4 hours (FIG. 4-11). At the same condition, no appreciable impurity peaks were observed for the Geneoxl-PM placebo sample.

### Acid Degradation

It was found that paclitaxel content of Genexol-PM was decreased from 99.8% to 86.9% rapidly for 24 hours at 1 N HCl solution. FIG. 4-12 illustrates the HPLC-UV chromatogram of Genexol-PM incubated in the aqueous solution of 1 N HCl for 24 hours. The two primary degradation products elute prior to paclitaxel, indicating their polar characteristics versus paclitaxel. The earlier eluting degradant at 12.6 min (RRT 0.71) has the same relative retention time as 10-deacetylpaclitaxel. The impurity at RRT 0.71 had the most prominent peak area which increased from 0% to 9.6% for 24 hours. The mpurity at RRT 0.82 was also significantly increased from 0.05% to 2.36% for 24 hours. At the same condition, no appreciable impurity peaks were observed for the Geneoxl-PM placebo sample.

### Oxidation

It was found that paclitaxel content of Genexol-PM was decreased from 99.8% to 92.4% for 30 hours at 3% H₂O₂ solution. FIG. 4-14 illustrates the HPLC-UV chromatogram of Genexol-PM incubated for 30 hours. Only a single minor degradant was observed eluting prior to paclitaxel. This degradant was eluted at 14.5 min (RRT 0.83) increasing from 0% to 1.6% for 30 hours. Other impurities were negligible. Based on the minimal degradation observed with these oxidizing conditions, Genexol-PM appears to be relatively stable in an oxidizing environment. At the same condition, no appreciable impurity peaks were observed for the Geneoxl-PM placebo sample.

### Photolysis

Paclitaxel content of Genexol-PM was rapidly decreased from 99.8% to 91.6% for 15 days under the UV/Fluorescent lamp. The samples were exposed by UV and fluorescent lamp simultaneously for the initial 8 days. Thereafter, they were exposed only by fluorescent lamp. As a result, the paclitaxel contents were found to be 92.6% at Day 8 and 91.6% at Day 15 indicating that Genexol-PM is more sensitive to UV light than fluorescent light. The largest and most interesting degradant corresponded to the 12.1 min peak (RRT 0.68) which increased from 0% to 8.0% for 15 days. At the same condition, no appreciable impurity peaks were observed for the Geneoxl-PM placebo sample.

### Example 7

### Pharmacokinetics in Humans

As part of a Phase I trials the pharmacokinetic parameters of paclitaxel following a three hour infusion of Genexol-PM ( ) were estimated for dose levels of 135, 175, 230, 300 and 390 mg/m² in one study and 85, 175, 290 and 300 mg/m² in a second study.

TABLE 3
Pharamacokinetic Parameters of Paclitaxel after a 3-hour Constant Rate Infusion Study 1

| Dose | No. | | Tₘₐₓ | Cₘₐₓ | T_{1/2} | AUC_{fast} | AUC_{inf} | V_{d} | CL |
|---|---|---|---|---|---|---|---|---|---|
| (mg/m₂) | Subjects | | (hr) | (mg/mL) | (hr) | (nghr/mL | (nghr/mL | (L/m²) | (L/hr/m²) |
| 135 | 3 | Mean | 2.01 | 1357 | 12.7 | 4935 | 5473 | 704 | 25.5 |
| | | SD | 0.86 | 254 | 4.2 | 979 | 1297 | 35 | 5.3 |
| 175 | 3 | Mean | 1.67 | 1470 | 12.5 | 5433 | 5740 | 897 | 32.0 |
| | | SD | 0.30 | 208 | 3.0 | 1366 | 1391 | 145 | 8.8 |
| 230 | 2 | Mean | 3.22 | 4682 | 11.0 | 18860 | 19476 | 328 | 12.1 |
| | | SD | 0.00 | 1543 | 1.9 | 4175 | 4004 | 128 | 2.5 |
| 300 | 3 | Mean | 3.24 | 3107 | 11.4 | 11239 | 11580 | 818 | 29.3 |
| | | SD | 0.11 | 1476 | 2.4 | 4305 | 4277 | 341 | 13.8 |
| 390 | 2 | Mean | 3.19 | 6567 | 17.9 | 26705 | 27491 | 655 | 14.9 |
| | | SD | 0.17 | 1120 | 1.0 | 7905 | 8297 | 218 | 4.5 |

TABLE 4
Pharamacokinetic Parameters of Paclitaxel after a 3-hour Constant Rate Infusion Study 2

| Dose | No. | | Tₘₐₓ | Cₘₐₓ | T_{1/2} | AUC_{fast} | AUC_{inf} | V_{d} | CL |
|---|---|---|---|---|---|---|---|---|---|
| (mg/m₂) | Subjects | | (hr) | (mg/mL) | (hr) | (nghr/mL | (nghr/mL | (L/m²) | (L/hr/m²) |
| 83 | 3 | Mean | 2.08 | 714 | 16.5 | 2591 | 2790 | 753 | 31.0 |
| | | SD | 0.96 | 191 | 6.5 | 478 | 449 | 342 | 5.3 |
| 175 | 3 | Mean | 1.67 | 1763 | 11.1 | 6948 | 8016 | 406 | 23.3 |
| | | SD | 1.11 | 520 | 5.7 | 2216 | 2841 | 325 | 8.3 |
| 290 | 5 | Mean | 1.45 | 2232 | 14.3 | 9571 | 9940 | 620 | 33.3 |
| | | SD | 0.86 | 694 | 7.2 | 3574 | 3808 | 186 | 13.7 |
| 435 | 8 | Mean | 2.15 | 4675 | 16.6 | 21673 | 22066 | 497 | 20.6 |
| | | SD | 0.96 | 823 | 4.0 | 4823 | 4905 | 165 | 4.5 |

Mean pK parameters of paclitaxel at dose levels 175, 290 and 300 mg/m² from the two studies are shown in the Table below:

TABLE 5
Pharamacokinetic Parameters (mean) of Paclitaxel after a 3-hour Infusion

| | Dose | No. | Cₘₐₓ | | AUC_{inf} | V_{d} | CL |
|---|---|---|---|---|---|---|---|
| Parameter | (mg/m²) | Subjects | (mg/mL) | T_{1/2} | (nghr/mL | (L/m²) | (L/hr/m²) |
| Study 1 | 175 | 3 | 1470 | 12.5 | 5740 | 897 | 32.0 |
| | 300 | 3 | 3107 | 11.4 | 11,580 | 818 | 29.3 |
| Study 2 | 175 | 3 | 1763 | 11.1 | 8016 | 406 | 23.3 |
| | 290 | 3 | 2232 | 14.3 | 9940 | 620 | 33.3 |

The studies indicate that Genexol-PM displayed an increased dose-proportionality over the ranges of 85-435 mg/m² indicating predictable linear pharmacokinetics.

The PK parameters of paclitaxel from Genexol-PM studies demonstrated a low degree of variability and increased dose proportionally over the dose range tested and were quite different from paclitaxel PK for Taxol reported in the literature. The mean paclitaxel concentration-time profiles following an infusion of Genexol-PM were characterized by a pronounced distribution phase followed by the terminal elimination phase. Within 5 to 15 minutes of the end of the infusion, paclitaxel concentrations dropped to one-half or one-third of the peak level. Following a constant-rate infusion of Genexol-PM, maximum plasma paclitaxel concentrations were observed between 1.45 and 3.22 hours after the start of the infusion. Differences between the observed Tmax values and the predicted value of 3 hours (the duration of the infusion) may be due to the variability in the actual infusion rate/duration and the timing of sample collection with respect to the end of the infusion. Mean values of Cmax ranged from 714 after 85 mg/m2 to 6,567 after 390 mg/m2. These values are somewhat lower than maximum paclitaxel concentrations observed after the administration of Taxol. A 3-hour Taxol infusion of 135 mg/m2 produced a mean maximum paclitaxel concentration 1.6 times greater than the mean Cmax value observed after a comparable dosing regimen of Genexol-PM in GXLPM. At the 175 mg/m2 dose level, there were 2.1-2.5 fold differences between paclitaxel Cmax values the administration of Taxol and Genexol-PM. Mean values of the paclitaxel half-life after the administration of Genexol-PM ranged from 11.0 to 17.9 hours which were consistent with those reported in the product label for Taxol (13 hours for 135 mg/m2 and 20 hours for 175 mg/m2).4 Mean values of the total area under the curve (AUCinf) ranged from 2,790 to 27,491 ng·hr/mL over the dose range tested. The mean AUC values allowed a direct comparison of systemic exposures between Genexol-PM and Taxol. Greater systemic exposures were observed after Taxol than after comparable dosing regimens of Genexol-PM. For the 135, 175, and 230/225 mg/m2 dose levels, the AUC values obtained after Taxol were 1.5-2.6 times greater than those obtained after Genexol-PM. The mean values of total systemic clearance of paclitaxel following 3-hour infusions of Genexol-PM were 12.1-33.3 L/hr/m2.4, 16 These values found to be somewhat higher than the systemic total body clearance published in the literature for Taxol (12.2 to 17.7 L/hr/m2).4 The mean Vd of paclitaxel in the terminal elimination phase following infusions of Genexol-PM ranged from 328 to 897 L/m2, which is significantly greater than the Vd of paclitaxel reported for Taxol (67 to 182 L/m2: estimated from mean values of CL and T½).

The smaller Cmax values observed after Genexol-PM administration in comparison to the Cmax values after similar doses of Taxol may be explained by differences in clearance between the two formulations and the larger volume of distribution of Genexol-PM. Paclitaxel administered as Genexol-PM appears to be cleared from the systemic circulation faster than comparable paclitaxel doses administered as Taxol. It is possible that the formulation influences tissue partitioning, which indirectly alters the accessibility of paclitaxel to metabolizing enzymes and estimates of clearance. Previous animal studies have shown that the Genexol-PM formulation increases paclitaxel tissue distribution relative to Taxol. Thus, the increase in the clearance estimates of paclitaxel when administered as Genexol-PM may be due to the removal of paclitaxel from systemic circulation by the pronounced binding to peripheral tissues. Although the binding in tissues is reversible and paclitaxel eventually reenters the plasma, the concentration-time data in these studies indicate that the flux of paclitaxel from the periphery into the central compartment is slow during the elimination phase. In accordance with the above discussion of clearance, paclitaxel administered as Genexol-PM was found to have a large volume of distribution, indicating extensive extra vascular distribution and/or a high degree of binding to tissues in the periphery relative to plasma protein binding. The Vd values estimated from the mean CL and T½ values of previous Taxol studies were consistently smaller than those calculated from Genexol-PM, suggesting that the Genexol-PM formulation produces greater distribution to the periphery in comparison to Taxol. From the results of the studies of Genexol-PM and the results from previous studies of Taxol, there appears to be some degree of inter-subject variability in the PK of paclitaxel, although this was much apparent in Taxol. Possible sources for the PK variability reported for paclitaxel include the wide intersubject differences in CYP3A and CYP2C8 enzyme systems, 3-4 saturable distribution processes, and differences in experimental design, namely the duration of the sampling interval and the detection of the actual terminal half-life. In addition, the magnitude of Vd is likely related to the amount of body fat in the subject since paclitaxel is highly lipophilic. Thus, body type could be another source for PK variability of paclitaxel regardless of the formulation.

Within this disclosure, any indication that a feature is optional is intended provide adequate support (e.g., under 35 U.S.C. 112 or Art. 83 and 84 of EPC) for claims that include closed or exclusive or negative language with reference to the optional feature. Exclusive language specifically excludes the particular recited feature from including any additional subject matter. For example, if it is indicated that A can be drug X, such language is intended to provide support for a claim that explicitly specifies that A consists of X alone, or that A does not include any other drugs besides X. “Negative” language explicitly excludes the optional feature itself from the scope of the claims. For example, if it is indicated that element A can include X, such language is intended to provide support for a claim that explicitly specifies that A does not include X. Non-limiting examples of exclusive or negative terms include “only,” “solely,” “consisting of,” “consisting essentially of,” “alone,” “without”, “in the absence of (e.g., other items of the same type, structure and/or function)” “excluding,” “not including”, “not”, “cannot,” or any combination and/or variation of such language.

Similarly, referents such as “a,” “an,” “said,” or “the,” are intended to support both single and/or plural occurrences unless the context indicates otherwise. For example “a dog” is intended to include support for one dog, no more than one dog, at least one dog, a plurality of dogs, etc. Non-limiting examples of qualifying terms that indicate singularity include “a single”, “one,” “alone”, “only one,” “not more than one”, etc. Non-limiting examples of qualifying terms that indicate (potential or actual) plurality include “at least one,” “one or more,” “more than one,” “two or more,” “a multiplicity,” “a plurality,” “any combination of,” “any permutation of,” “any one or more of,” etc. Claims or descriptions that include “or” between one or more members of a group are considered satisfied if one, more than one, or all of the group members are present in, employed in, or otherwise relevant to a given product or process unless indicated to the contrary or otherwise evident from the context.

Where ranges are given herein, the endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While this invention has been particularly shown and described with references to example embodiments thereof, it will be understood by those skilled in the art that the various changes in form and details may be made therein without departing from the scope of the invention encompassed by the appended claims.

Further advantages of the present immunological compositions and adjuvants of the present invention can be achieved by those skilled in the art based upon the embodiments described herein and are thus specifically within the scope of the present invention.

1. A composition comprising a polymeric formulation of paclitaxel encapsulated in monomethoxy poly(ethylene glycol)-block-poly(D,L-lactide) in a nanoparticle wherein the wherein the monomethoxy poly(ethylene glycol)-block-poly(D,L-lactide) has a molecular weight of about 3000-4000 and wherein the nanoparticles are from about 10 nm to about 50 nm composition is unstable in the human body.

2. The composition of claim 1 wherein the paclitaxel concentration is sufficient to administer to a patient a dose of from about 50 mg/m² to 500 mg/m² of paclitaxel

3. The composition of claim 2 wherein the loading efficiency of paclitaxel is from about 95.0% to about 99.9%.

4. The composition of claim 2 wherein the loading efficiency of paclitaxel is from about 97.0% to about 99.9%.

5. The composition of claim 1 wherein the loading efficiency of paclitaxel is from about 98.0% to about 99.9%.

6. The composition of claim 1 wherein the nanoparticles are from about 20 nm to about 50 nm.

7. The composition of claim 1 wherein the nanoparticles are from about 20 nm to about 30 nm.

8. The composition of claim 1 wherein the polydispersity index of the nanoparticles is about 0.120 to 0.145).

9. The composition of claim 1 wherein the composition is stable outside a human body and wherein the composition is unstable in the human body and wherein the composition is bound to and transported by endogenous protein.

10. The composition of claim 1 wherein the nanoparticles are made by a method comprising:
melting the mPEG-PDLLA in a mixing tank for more than 30 minutes;
adding paclitaxel and anhydrous ethanol to the mPEG-PDLLA and mixed for about 30 to about 35 minutes;
evaporating the ethanol is evaporated under vacuum for about 120 minutes; and
adding water for injection mixing for about 40 to about 60 minutes to produce the nanoparticles.

11. The composition of claim 7, the method further comprising preparing an anhydrous lactose and water for injection solution and adding the nanoparticles.

12. The composition of claim 1 wherein the surface charges range from about −2.0 mV to about +1.0 mV.

13. The composition of claim 1 wherein the critical micelle concentration of the composition is about 0.007 mg/mL.

14. The composition of claim 1 wherein the composition is stable outside a human body and wherein the composition is unstable in the human body.

15. The composition of claim 1 wherein the composition is stable in a non-protein containing medium and wherein the composition is unstable protein containing medium.

16. The composition of claim 1 wherein the composition is stable in a non-protein containing medium and wherein the composition is unstable protein containing medium.

17. A method of treating cancer patients comprising administering to the patient the composition of claim 1 at a paclitaxel dosage of between 135 mg/m² to 390 mg/m² wherein the administration of the composition is performed by infusion over a three hour period yields a Cₘₐₓ of between about 1000 to about 7000 ng/ml and a Tₘₐₓ of between about 1 hour to about 4 hours and a T_{1/2} of between about 10 hours to about 18 hours and an AUC_{inf} of between about 4000 to about 27000 nghr/ml.

18-20. (canceled)

21. A method of treating cancer patients comprising administering to the patient the composition of claim 1 at a paclitaxel dosage of between 85 mg/m² to 435 mg/m² wherein the administration of the composition is performed by infusion over a three hour period yields a Cₘₐₓ of between about 500 to about 7000 ng/ml and Tₘₐₓ of between about 1 hour to about 4 hours and a T_{1/2} of between about 10 hours to about 18 hours and an AUC_{inf} of between about 4000 to about 27000 nghr/ml.

22-25. (canceled)

26. A method of treating cancer patients comprising administering to the patient the composition of claim 1 at a paclitaxel dosage of between 85 mg/m² to 435 mg/m² wherein the administration of the composition is performed by infusion for about 15 minutes to about 45 minutes.

27. The method of claim 27 wherein the administration of the composition is performed by infusion for about 30 minutes.
